(19) United States
(12) Patent Application Publication
(10) Pub. No.: US 2003/0120322 A1
(43) Pub. Date: Jun. 26, 2003

(54) Neurophysiologic basis of idiopathic diseases

(76) Inventor: Anuthep Benja-Athon, New York, NY (US)

Correspondence Address: ANUTHEP BENJA-ATHON, GR. FLOOR, 210 E 36TH ST., NEW YORK, NY, 10016, US

(21) Appl. No.: 10/274,534

(22) Filed: Oct. 21, 2002

Related U.S. Application Data
(63) Continuation-in-part of application No. 08/851,181, filed on May 5, 1997, now Pat. No. 5,861,015.
(63) Continuation-in-part of application No. 09/884,025, filed on Jun. 20, 2001, now abandoned.

(51) Int. Cl. A61N 1/18

(52) U.S. Cl. 607/46

(57) ABSTRACT

A discovery and elucidation of the neurophysiologic basis of idiopathic rheumatism, degenerative and auto-immune diseases and disorders in a human in whom an injury to, a disorder or derangement of the participant or participants comprise the tissues of the spine, organs peripheral to the spinal tissues, small, medium and large, unmyelinated and myelinated afferent neurons, preganglionic sympathetic neurons, postganglionic sympathetic neurons and interneurons initiates a cascade or cascades of events leading to idiopathic diseases and disorders. The cascades of events are the permutations involving one participant or two or more participants. The injured and disordered tissues formed at the end of a cascade of events may become the antigenic “foreign bodies” which are recognized and processed by the immune system leading to the auto-immune diseases and disorders.

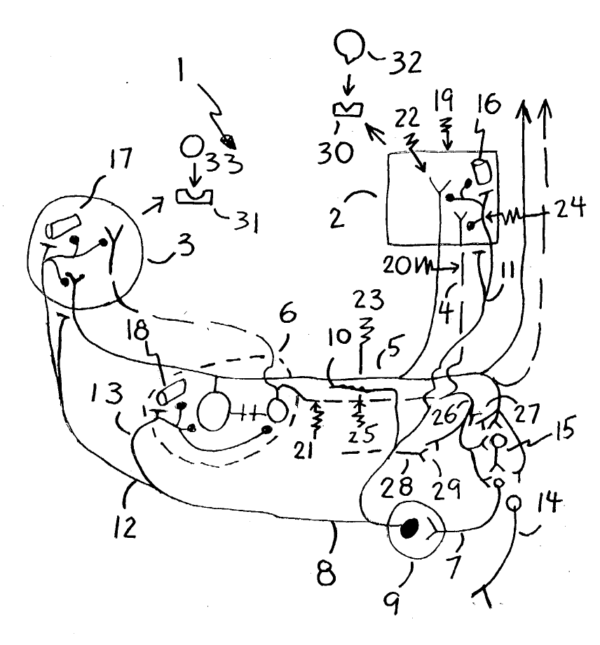

This is a continuation-in-part application of U.S. Pat. No. 5,861,015 granted to this applicant on Jan. 19, 1999 entitled Modulation of the Nervous Systems for Treatment of Pain and Related Disorders.

### FIELD OF INVENTION

A discovery and an elucidation of the etiology, pathogenesis and biomechanism of human diseases bearing such labels as “idiopathic” and “unclear or unknown pathogenesis” and the like.

### BACKGROUND OF THE INVENTION

This patent applicant filed a patent application Ser. No. 09/320,745 on Mat 27, 1999, now abandoned, entitled Computerized Method for Depicting the Origin of Organ Disorders. This patent applicant also filed a first continuation-in-part application Ser. No. 09/884,025 entitled the Neurophysiologic Basis of Idiopathic Diseases on Jun. 20, 2001 which was abandoned.

The present invention is the discovery and illustration of the neurophysiologic basis of many idiopathic diseases and disorders bearing the label of unknown or unclear etiology, cause, pathogenesis or mechanism.

The objective is to elucidate, demonstrate and describe the portion of the neurophysiology, pathogenesis and mechanism of many idiopathic diseases and disorders which was not described or briefly stated in the U.S. Pat. No. 5,861,015 entitled Modulation of the Nervous Systems for Treatment of Pain and Related Disorders granted to this applicant.

The other objective is to lead to and support further researches and medical advancements in the discovery the preventions, effective treatments and cures for many common diseases and disorders afflicting humans.

To support U.S. Pat. No. 5,861,015, the following concepts were stated throughout said U.S. Pat. No. 5,861,015 and are briefly represented herein as follow: In U.S. Pat. No. 5,861,015, this applicant described a technique of treating pain and inflammations including neurogenic inflammation. Said medical method was based on the applicant's brief definition of an “Innervation Unit: Any parts and organs in an Innervation Unit having same, related, linked, connected, coupled anatomy, neuroanatomy and neurophysiology by and in the peripheral nervous system, central nervous system, or both.” (Column 1, lines 39-44)

In said patent, applicant stated that “Many of the pain and inflammation conditions including neurogenic inflammation and related disorders of visceral and somatic organs involve, are mediated and perpetuated by the afferent nervous system and autonomic nervous system are responsive to treatment method of the present invention. It will be discovered that many of the pain and inflammations and related disorders of the visceral and cardiovascular organs which elude scientific explanation and treatment will follow the scheme of the present invention.” (Column 1, lines 28-37) Many of said pain and inflammations involve and mediate by the afferent of the sensory nervous system and the afferent of the autonomic nervous system and, subsequently, involved the efferent nervous system of the autonomic nervous system and the motor nervous system” (Column 1, lines 51-56)

Aging, degeneration, pain, rheumatism and inflammations, paralysis, autoimmune and related diseases and disorders are common diseases afflicting all humans and animals. Until now, the etiologies, causes, pathogeneses and mechanisms of these diseases and disorders remain either unclear or unknown. Said diseases and disorders commonly are labeled “idiopathic”. These diseases and disorders are represented herein by repetitive strain injuries, cumulative trauma disorders, fibromyalgia, soft tissue diseases, myalgia, myositis, myofascial pain syndromes including myofascial pain, enthesopathies such as tendinitis, bursitis, chronic fatigue syndrome, inflammation of soft tissues, rheumatism including palindromic rheumatism, arthropathies including degenerative osteoarthritis and rheumatoid arthritis, neuropathy includes neuritis, neuralgia, nerve entrapment, Bell's Palsy, chronic pain syndrome including referred pain syndrome, disorders of the autonomic nervous system including reflex sympathetic dystrophy, skin disorders such as psoriasis, enteropathy, gastrointestinal, cardiopathy, vasculopathy and cardiovascular diseases such as, but not limited to colitis, irritable bowel and various inflammatory diseases of the gastrointestinal tract and organs, heart and large, medium and small blood vessels, lymphatic system, and autoimmune diseases such as, but not limited to, multiple sclerosis.

Consequently, the treatments and cures remain elusive. Humans continue to endure untold suffering. The avoidable morbidity and untimely mortality stilt prevail. The tangible cost to the society alone is in the hundreds of billions of dollars every year. However, the discovery and elucidation being presented in this patent application will certainly lead to the discoveries of treatments, cures and preventions of said diseases and disorders.

### SUMMARY OF THE INVENTION

The etiology, cause, pathogenesis and biomechanism of many common diseases and disorders remain either unclear or unknown.

In U.S. Pat. No. 5,861,015 entitled Modulation of the Nervous Systems for Treatment of Pain and Related Disorders granted to this applicant, only a portion of the neurophysiology pertinent to the patent technique was briefly mentioned thereby providing a scientific basis of the patented technique for treating pain and inflammations including neurogenic inflammation.

The present invention is a discovery and elucidation of the portion of the neurophysiologic basis which forms the fundamental of the origin and cause of the idiopathic rheumatisms, degenerative and auto-immune diseases and disorders in a human. In an individual, an injury to, a disorder or derangement of a participant, two or more participants comprise the tissues of the spine, organs peripheral to the spinal tissues, small, medium and large, unmyelinated and myelinated afferent neurons, preganglionic sympathetic neurons, postganglionic sympathetic neurons and interneurons initiates a cascade or cascades of events leading to said and many other idiopathic diseases and disorders. The cascade or cascades of events are numerous permutations involving one participant or two or more participants. The injured and disordered tissues formed at the end of each cascade of events may become the antigenic “foreign bodies” which are recognized and processed by the immune system leading to cell-mediated and humoral immunity thence the chain interaction between the immune system and said related participant's or participants' tissues. A result is the auto-immune diseases and disorders.

### BRIEF DESCRIPTION OF THE DRAWING

A schematic illustration of the present invention.

### PREFERRED EMBODIMENTS OF THE INVENTION

The drawing is a schematic representation of the present invention which is a discovery and an elucidation of the neurophysiologic basis of rheumatisms, degenerative and autoimmune diseases and disorders 1 in humans and animals. The neurophysiologic basis of said diseases and disorders 1 comprises the interaction between the participating tissues such as, but not limited to, tissues of the spine 2, organs 3 peripheral to spinal tissues 2, small unmyelinated and myelinated afferent neurons 4 which are shown as broken lines in the drawing, medium and large, unmyelinated and myelinated afferent neurons 5, cell bodies (clear circles) in peripheral ganglia 6, preganglionic sympathetic neurons (PESN) 7 in the spinal cord, postganglionic sympathetic neurons (PSN) 8 and their extensive and ubiquitous axons 10, 11, 12 and 13, endings of PSN 8 as represent by the arbors of dark circles in spinal tissues 2, organs 3 and dorsal root ganglia 6, cell bodies of PSN 8 in the sympathetic ganglia 9, motoneurons 14, interneurons 15, and the immune system. Afferent neurons 4 and 5 comprising axons, central and peripheral endings, receptors, cell bodies (clear circles) in dorsal root ganglia 6, dendrites and dendrodendritic bundles (two “+'s” in ganglia 6).

Spinal tissues 2 comprises muscles, joints, nerves, ligaments, tendons, sheaths, cartilage, discs, connective tissues, blood vessels, lymphatics and bones and their microscopic components. The organs 3 peripheral to spinal tissues 2 are such as, but not limited to, the nervous, musculoskeletal, cardiovascular, lymphatic, blood, gastrointestinal, visceral, integument and endocrine systems and their microscopic components.

Small unmyelinated and myelinated afferent neurons 4 are represented by a broken line and medium and large unmyelinated and myelinated afferent neurons 5 are represent by a solid line. Afferent neurons 4 and 5 include A-alpha, A-beta, A-gamma, A-delta and C nerve fibers. Moreover, afferent neurons adaptable to transmit pain and nociceptive sensations also travel with PSN 8 to reach the spinal cord and supraspinal level (not shown). The cell bodies of these neurons are in dorsal root ganglia 6 and other peripheral ganglia.

The axons of small unmyelinated and myelinated afferent neurons 4 and medium and large, unmyelinated and myelinated afferent neurons 5 are shown to associate with, relate to and/or innervate spinal tissues 2 and organs 3 as represent by “Y”'s in tissues 2 and organs 3.

The drawing also shows the extensive and ubiquitous projection of axons 10, 11, 12 and 13 of PSN 8. Axons 10 of PSN 8 are associate with afferent neurons 4 and 5, axons 11, 12 and 13 reach tissues 2 and organs 3 by several means. One of the means for reaching tissues 2 and organs 3 is for said axons to travel with blood vessels 16, 17 and 18.

The following discovery elucidates the origin and biomechanism of many diseases and disorders labeled “unknown etiology, pathogenesis or biomechanism” and the like.

In a first setting, a cascade of events ensue after an injury to or a disorder of any said participating tissue such as, but not limited to: a) spinal tissues 2 such as, but not limited to, muscles, joints and/or ligaments as represents by wavy arrow 19, b) afferent neurons 4 within or adjacent to spinal tissues 2 as represents by wavy arrow 20 or distant to spinal tissues 2 as represent by wavy arrow 21, c) afferent neurons 5 within or adjacent to spinal tissues 2 as represents by wavy arrow 22 or distant to spinal tissues 2 as represents by wavy arrow 23, or d) axons II of PSN 8 within or adjacent to spinal tissues 2 as represents by wavy arrow 24 or axons 10 of PSN 8 distant to spinal tissues 2 as represents by wavy arrow 25.

In this setting, a cascade of events involving said participating tissues ensues. For example, taking (a) in said first setting, the neurophysiologic origin and cause of said diseases and disorders such as, but not limited to, fibromyalgia, cervicobrachial pain syndrome, myofascial pain syndrome, trigger point and palindromic rheumatism is an injury to, a disorder or derangement of participating spinal tissues 2 such as, but not limited to, ligaments, muscles joints and nerves in the spine. Associate with or related to these injured and disordered tissues 2 are afferent neurons 4, afferent neurons 5 and axons 11 of PSN 8. As a result, afferent neurons 4, afferent neurons 5, PSN 8 or any combination thereof are activated or sensitized by the chemicals released by the injured or disordered tissues 2. In a first subset, afferent neurons 4 are activated or sensitized by said released chemicals from the injured or disordered cells of spinal tissues 2. The central axons and endings 26 of said activated or sensitized afferent neurons 4 activate or sensitize the central portion of afferent neurons 5 in the spinal cord at 27 and/or supraspinal level (not shown). Said activation or sensitization may be directly via synapses or via interneurons (not shown). The result is the activation or sensitization of entire afferent neurons 5 including their cell bodies in ganglia 6, axons, the peripheral endings and receptors as represent by bold “Y” in distant organs 3. Some said afferent neurons 5 also innervate tissues 2. In a second subset, afferent neurons 4 are activated or sensitized by said released chemicals from the injured or disordered cells of spinal tissues 2. The central axons and endings 26 of said activated or sensitized afferent neurons 4 activate or sensitize the central portion of other afferent neurons 28 in the spinal cord at 29 and/or supraspinal level (not shown). The result is the activation or sensitization of entire other afferent neurons 28 including their cell bodies in ganglia 6, axons, the peripheral endings and receptors distant organs 3. Some said afferent neurons 5 also innervate tissues 2. In a third subset, afferent neurons 4 are activated or sensitized by said released chemicals from the injured or disordered cells of spinal tissues 2. The central axons and endings 26 of said activated or sensitized afferent neurons 4 activate or sensitize PSN 8 via supraspinal neurons (not shown), PESN 7 directly or via interneurons 15 in the spinal cord. A result is the activation or sensitization of PSN 8 including their extensive and ubiquitous axons 10, 11, 12, and 13 and their endings “T's” and arbors of dark circles in spinal tissues 2, organs 3 and ganglia 6. In this third subset, PSN 8 activate or sensitize cell bodies in dorsal root ganglia 6, axons, said endings and receptors of afferent neurons 4 and 5 in tissues 2 and organs 3. PSN X further affect and modulate the activities of other local tissues of spinal tissues 2 and organs 3 such as, but not limited to, the blood vessels, lymphatics and endocrine organs.

The above three subsets serve as examples for other permutations resulting in cascades of events involving afferent neurons 4, afferent neurons 5 or PSN 12 in (b), © and (d) supra, respectively. For example, in afferent neurons 4 in (b) supra are injured or deranged as represent by wavy line 20. Consequently, other afferent neurons 4, afferent neurons 5 or PSN 8 and any combination thereof are activated or sensitized by the chemicals being released by the injured or disordered afferent neurons 4 in the peripheral nervous system and central nervous system. In this first subset, the central axons and endings 26 of injured or disordered afferent neurons 4 activate or sensitize the central portion of other afferent neurons 28 in the spinal cord at 29 or supraspinal level (not shown). Said interaction may be via direct synapses and/or interneurons. The result is the activation or sensitization of entire afferent neurons 28 including their cell bodies, axons, the peripheral endings and receptors in distant organs 3. Some said afferent neurons 28 also innervate tissues 2. In this second subset, central axons and endings 26 of said injured or disordered afferent neurons 4 activate or sensitize the central portion of afferent neurons 5 at 27 at the spinal cord or supraspinal level. The result is the activation or sensitization of entire afferent neurons 5 including their cell bodies, axons, the peripheral endings and receptors, latter are represented by bold and enlarged “Y” in distant organs 3. Some said afferent neurons 5 also innervate tissues 2. In this third subset, the central axons and endings 26 of said injured or disordered afferent neurons 4 activate or sensitize PESN 7 directly or via interneuron 29 in the spinal cord or supraspinal level. The result is the activation or sensitization of entire PESN 7 and PSN 8 including their axons 10, 11, 12 and 13 and endings as represent by the arbor of “T” and dark circles in spinal tissues 2, organs 3 and ganglia 6. PSN 8 have the effect of activating or sensitizing cell bodies in ganglia 6, axons, endings and receptors of afferent neurons 4, 5 and 28. PSN 8 further affect and modulate the activities of other local tissues of spinal tissues 2 and organs 3.

In a second setting, a cascade of events ensue after an injury to, a disorder or derangement of two or more said participating tissues at a given time, such as, but not limited to, any combination of two or more of the following: aa) the spinal tissues 2 as represents by the wavy arrow 19, bb) afferent neurons 4 within or adjacent to spinal tissues 2 as represents by wavy arrow 20 or distant to spinal tissues 2 as represent by wavy arrow 21, cc) afferent neurons 5 within or adjacent to spinal tissues 2 as represents by wavy arrow 22 or distant to spinal tissues 2 as represents by wavy arrow 23, dd) PSN 8 within or adjacent to spinal tissues 2 as represents by wavy arrow 24 or PSN 8 distant to spinal tissues 2 as represents by wavy arrow 25.

In the first setting, for example, taking participating tissues (aa) and (bb) supra, the neurophysiologic origin and biomechanism of fibromyalgia, cervicobrachial pain syndrome, myofascial pain syndrome and trigger point elucidate an injury to or a disorder of participating spinal tissues 2 such as, but not limited to, ligaments, muscles, joints and nerves in the spine and, concomitantly, afferent neurons 4. Associate with or related to these injured and disordered tissues 2 and afferent neurons 4 are other afferent neurons 28, afferent neurons 5 and PSN 8. As a result, afferent neurons 28, afferent neurons 5, PSN 8 or any combination thereof are activated and sensitized by the chemicals being released by the injured or disordered tissues 2 and afferent neurons 4. In a first subset, the central axons and endings 26 of said injured or disordered afferent neurons 4 activate or sensitize the central portion of other afferent neurons 28 and afferent neurons 5 in the spinal cord at 27 and/or supraspinal level. The result is the activation or sensitization of entire other afferent neurons 28 and afferent neurons 5 including their cell bodies in ganglia 6, axons, the peripheral endings and receptors as represented by “Y's” in spinal tissues 2 and distant organs 3. In a second subset, the central axons and endings 26 of injured or disordered neurons 4 activate or sensitize other afferent neurons 28, afferent neurons 5, PSN 8 via PESN 7 and other centers in the spinal cord or supraspinal level. The result is the activation or sensitization of entire other afferent neurons 28, afferent neurons 5 and PSN 8 including their axons and endings and receptors as represent by the “Y's” in tissues 2 and organs 3 and the arbor of “T's” and dark circles in spinal tissues 2, organs 3 and ganglia 6. Moreover, PSN 8 activates or sensitizes cell bodies in ganglia 6, axons, endings and receptors of afferent neurons 4 and 5. PSN 8 further affect and modulate the activities of other local tissues of spinal tissues 2 and organs 3.

Other settings, the above three subsets serve as examples for other cascades of events involving any combination of two or more participating tissues in (aa), (bb), (cc) and (dd) supra. For example, a combination of (bb) and (dd) involve injury or disorder of afferent neurons 4 and axons 10 or 11 of PSN 8 as represent by wavy lines 25 and 24, respectively. Consequently, other afferent neurons 28, afferent neurons 5, other axons 12 and 13 of PSN 8 are activated and sensitized by the chemicals released by the injured or disordered afferent neurons 4 in the peripheral nervous system and/or central nervous system and/or by chemicals released by PSN 8. In a first subset, the central axons and endings 26 of other afferent neurons 4 are activated or sensitized the central portion of afferent neurons 28 in the spinal cord at 29 or supraspinal level. The result is the activation or sensitization of entire afferent neurons 28 including their cell bodies, axons, the peripheral endings and receptors in distant organs 3 or spinal tissues 2. In a second subset, central axons and endings 26 of said injured or disordered afferent neurons 4 activate or sensitize the central portion of afferent neurons 5 at 27 at the spinal cord or supraspinal level. The result is the activation or sensitization of entire afferent neurons 5 including their cell bodies, axons, the peripheral endings and receptors, latter are represented by bold and enlarged “Y” in distant organs 3 or spinal tissues 2. In a third subset, the central axons and endings 26 of said injured or disordered afferent neurons 4 activate or sensitize PSN 8 via PESN 7 and other spinal cord or supraspinal centers. The result is the activation or sensitization of entire PESN 7 and PSN 8 including their axons and endings and receptors as represent by the arbor of “T” and dark circles in spinal tissues 2, organs 3 and ganglia 6. PSN 8 have the effect of activating or sensitizing cell bodies, axons, endings and receptors of afferent neurons 4 and 5. PSN 8 further affect and modulate the activities of other local tissues of spinal tissues 2 and organs 3. In a fourth subset, included the above three subsets is the injured or sensitized PSN 8 which activate or sensitize cell bodies in ganglia 6, axons, endings and receptors of afferent neurons 4, 5 and 28 in organs 3 and spinal tissues 2.

For example, more narrowly, in said diseases and disorders, said injuries or disorders imposed on the soft tissues 2 of the cervical spine or lumbosacral spine 2 such as ligaments, muscles, joints, afferent neurons 4, afferent neurons 5 and/or PSN 8 cause the release of chemicals to activate or sensitized a first group of A-alpha, A-beta, A-gamma, A-delta and/or C fibers associate with, relate to or innervate soft tissues 2 Injured or disordered A-alpha, A-beta, A-gamma, A-delta and/or C fibers project axons 26 to the spine and supraspinal centers wherein they interact via synapses and interneurons with the central endings of other A-alpha, A-beta, A-gamma, A-delta and/or C fibers, PESN 7 and other spinal and supraspinal neurons. In a first subset, A-beta, A-gamma, A-delta and/or C fibers can be involved leading to the cascades of events supra. In n second subset, A-beta, A-gamma, A-delta, C fibers and/or PSN 8 arc involved leading to the cascades of events supra. In a third subset, A-alpha, A-beta, A-gamma and/or PSN 8 are involved leading to the cascades of events supra. Other subsets based on the all possible permutations using all participating tissues can be had. A result of this activation or sensitization is the activation or sensitization of A-alpha, A-beta, A-gamma, A-delta, C fibers and/or PSN 8 whose axons, endings and receptors are associate with, relate to or innervate distant organs 3 or spinal tissues 2 as described supra. PSN 8 can activate or sensitize cell bodies, axons, endings and receptors of A-beta, A-gamma, A-delta and C fibers. PSN 8 further affect and modulate the activities of other local tissues of spinal tissues 2 and organs 3.

Moreover, motoneurons 14 and other neurons in the spinal and other supraspinal centers such as, but not limited to, those centers in the brain stems and higher centers are affected and modulated by aforementioned participating tissues, cascades of events and phenomena. A result is the illnesses, manifestations and syndrome describing and associating with said diseases and disorders.

A consequence of the above cascades of events is formation of injured tissues and cells 30 and 31 derived from said participating tissues. A byproduct of said cascades of events, injured tissues and cells 30 and 31 are presented and processed by the cells of the cellular immune system such as, but not limited to, the macrophages, T- and B-cells and also by the antigen-antibody means for mounting a humoral immune response as represent by 32 and 33. In other words, the immune system recognizes injured tissues and cells 30 and 31 which are the byproduct of said cascades of events are recognized by the immune system as “foreign” particles. The immune system then mounts a cell-mediated and humoral immune reaction against tissues and cells 30 and 31. A consequence is the native tissue(s) and cell(s) of said participant or participants are also being attacked by the sensitized immune system leading to the auto-immune diseases and disorders.

The aforementioned phenomena and cascades of events are the representations of all possible permutations of events involving said participating tissues. They form the neurophysiologic basis explaining and elucidating the origin and biomechanism of many idiopathic diseases and disorders such as, but not limited to, fibromyalgia, cervicobrachial pain syndrome, myofascial pain syndrome, trigger points, palindromic rheumatism, arthropathies and other diseases and disorders mentioned supra.

Said injuries and disorders are such as, but not limited to, sprains, strains, degenerations, tears, infections, organ structural or cellular disruptions.

The dendrodendritic interaction can contribute to said phenomena.

Said activation or sensitization means that afferent neurons 4, 5, 28, PESN 7 and PSN 8, spinal and supraspinal neurons have lowered threshold, are more easily excitable, or are subjected to an alteration of the excitation and inhibition.

Although the preferred embodiments have been described, it will be appreciated by those skilled in the science and medicine that variations, permutations, sequences and orders of events and phenomena described herein may and do occur among diseases and disorders without departing from the spirit of the invention and the scope of the claims.

Although the preferred embodiments have been described for aforementioned diseases and disorders, it will be appreciated by those skilled in the science and medicine that said preferred embodiments, variations, permutations, sequences and orders of events and phenomena apply to other diseases and disorders which were neither described in nor implied by this patent application without departing from the spirit of the invention and the scope of the claims.

1. A discovery of the neurophysiologic basis of rheumatism, degenerative and auto-immune diseases and disorders in a human in whom an injury to, disorder or derangement of the participating tissue or tissues leads to a cascade of events wherein said participating tissues are the tissues of the spine, organs peripheral to said spinal tissues, small, medium and large, unmyelinated and myelinated afferent neurons, preganglionic sympathetic neurons (PESN), postganglionic sympathetic neurons (PSN), interneurons, spinal neurons and immune system and wherein said participating tissues are the participants in the production of said diseases and disorders comprises:
an injury to said participant; a disorder of said participant;
an injury to said participants; a disorder of said participants;
an activation of said afferent neurons associate with said participant;
a sensitization of said afferent neurons associate with said participant;
an activation of said afferent neurons innervating said participant;
a sensitization of said afferent neurons innervating said participant;
an activation of said afferent neurons associate with said participants;
an activation of said afferent neurons innervating said participants;
a sensitization of said afferent neurons innervating said participants;
an activation of the PSN by said participants;
a sensitization of PSN by said participants;
an activation of said injured afferent neurons including their cell bodies, axons, nerve endings and receptors, and dendrites by PSN;
a sensitization of said injured afferent neurons including their cell bodies, axons, nerve endings and receptors, and dendrites by PSN;
an activation of said disordered afferent neurons including their cell bodies, axons, nerve endings and receptors, and dendrites by PSN;
a sensitization of said injured afferent neurons including their cell bodies, axons, nerve endings and receptors, and dendrites by PSN;
an activation of a second group of afferent neurons by said injured participant;
an activation of a second group of afferent neurons by said disordered participant;
a sensitization of a second group of afferent neurons by said injured participants;
a sensitization of a second group of afferent neurons by said disordered participants;
an activation of a second group of afferent neurons including the activation of their cell bodies, axons, nerve endings and receptors, and dendrites associate with and by said disordered afferent neurons in the peripheral nervous system;
a sensitization of a second group of afferent neurons including the sensitization of their cell bodies, axons, nerve endings and receptors, and dendrites associate with and by said disordered afferent neurons in the peripheral nervous system;
an activation of a second group of afferent neurons including the activation of their cell bodies, axons, nerve endings and receptors, and dendrites associate with and by said activated afferent neurons in the peripheral nervous system;
a sensitization of a second group of afferent neurons including the sensitization of their cell bodies, axons, nerve endings and receptors, and dendrites associate with and by said sensitized afferent neurons in the peripheral nervous system;
a sensitization of a second group of afferent neurons associate with and by said activated afferent neurons in the central nervous system leading to the sensitization of the peripheral endings and receptors of said second group of afferent neurons in the peripheral nervous system;
an activation of a second group of afferent neurons relate to and by said sensitized afferent neurons in the central nervous system leading to the activation of the peripheral endings and receptors of said second group of afferent neurons in the peripheral nervous system;
an activation of a second group of afferent neurons associate with and by said disordered afferent neurons in the central nervous system leading to the activation of the peripheral endings and receptors of said second group of afferent neurons in the peripheral nervous system;
a sensitization of a second group of afferent neurons associate with and by said injured afferent neurons in the central nervous system leading to the sensitization of the peripheral endings and receptors of said second group of afferent neurons in the peripheral nervous system;
a sensitization of a second group of afferent neurons relate to and by said disordered afferent neurons in the central nervous system leading to the sensitization of the peripheral endings and receptors of said second group of afferent neurons in the peripheral nervous system;
an activation of PSN relate to said injured participant;
a sensitization of PSN relate to said disordered participant;
an activation of PSN relate to said injured participants;
a sensitization of PSN relate said disordered participants;
an activation of a second group of afferent neurons including the activation of their cell bodies, axons, nerve endings and receptors, and dendrites by PSN;
a sensitization of a second group of afferent neurons including the sensitization of their cell bodies, axons, nerve endings and receptors, and dendrites by PSN;
an activation of PESN by said participants;
a sensitization of the PESN by said participants;
an activation of said afferent neurons including the activation of their cell bodies, axons, nerve endings and receptors, and dendrites by injured PSN;
a sensitization of the afferent neurons including the activation of their cell bodies, axons, nerve endings and receptors, and dendrites by disordered PSN;
a means for sensitizing the immune system leading to said auto-immune diseases and disorders; and
a means for activating the immune system leading to said auto-immune diseases and disorders.

2. The discovery according to claim 1 wherein said activation of PSN by said participants includes the activation of PSN by injured and disordered afferent neurons.

4. The discovery according to claim 1 wherein said sensitization of PSN by said participants includes the activation of PSN by injured and disordered tissues of spinal tissues.

5. The discovery according to claim 1 wherein said sensitization of PSN by said participants includes the activation of PSN by injured and disordered tissues of organs peripheral to spinal tissues.

6. The discovery according to claim 1 wherein said an activation of a second group of afferent neurons associate with and by said sensitized afferent neurons in the central nervous system leads to a dorsal root reflex means for initiating said diseases and disorders.

7. The discovery according to claim 1 wherein said sensitization of a second group of afferent neurons associate with and by said activated afferent neurons in the central nervous system leads to a dorsal root reflex means for initiating said diseases and disorders.

8. The discovery according to claim 1 wherein said activation of a second group of afferent neurons relate to and by said sensitized afferent neurons in the central nervous system leads to a dorsal root reflex means for initiating said diseases and disorders.

9. The discovery according to claim 1 wherein said sensitization of a second group of afferent neurons relate to and by said sensitized afferent neurons in the central nervous system leads to a dorsal root reflex means for initiating said diseases and disorders.

10. The discovery according to claim 1 wherein said activation of said afferent neurons including their cell bodies, axons, nerve endings and receptors, and dendrites by the PSN involves the neurochemical and neuroendocrine means for interacting between said afferent neurons and PSN.

11. The discovery according to claim 1 wherein said sensitization of said afferent neurons including their cell bodies, axons, nerve endings and receptors, and dendrites by the PSN involves the neurochemical and neuroendocrine means for interacting between said afferent neurons and PSN

12. The discovery according to claim 1 wherein said activation of a second group of afferent neurons including their cell bodies, axons, nerve endings and receptors, and dendrites by the PSN involves the neurochemical and neuroendocrine means for interacting between said afferent neurons and PSN.

13. The discovery according to claim 1 wherein said sensitization of a second group of afferent neurons including their cell bodies, axons, nerve endings and receptors, and dendrites by the PSN involves the neurochemical and neuroendocrine means for interacting between said afferent neurons and PSN.

14. The discovery according to claim 1 wherein said means for sensitizing the immune system leading to said auto-immune diseases and disorders is an antigenic derivative of said injured or disordered participants.

15. The discovery according to claim 1 wherein said means for activating the immune system leading to said auto-immune diseases and disorders is an antigenic derivative of said injured or disordered participants.

16. A discovery of a neurophysiologic basis of rheumatism, degenerative and autoimmune diseases and disorders, specifically, fibromyalgia, fibrositis, myofascial pain syndrome, cervicobrachial pain syndrome, palindromic rheumatism, arthropathies, irritable gastrointestinal tract and idiopathic auto-immune soft tissue diseases in a human in whom an injury to, disorder or derangement of the participating tissue or tissues leads to a cascade of events wherein said participating tissues are the tissues of the spine, organs peripheral to said spinal tissues, small, medium and large, unmyelinated and myelinated afferent neurons, preganglionic sympathetic neurons (PESN), postganglionic sympathetic neurons (PSN), interneurons, spinal neurons and immune system and wherein said participating tissues are the participants in the production of said diseases and disorders comprises:
an injury to said participant; a disorder of said participant;
an injury to said participants; a disorder of said participants;
an activation of said afferent neurons associate with said participant;
a sensitization of said afferent neurons associate with said participant;
an activation of said afferent neurons innervating said participant;
a sensitization of said afferent neurons innervating said participant;
an activation of said afferent neurons associate with said participants;
an activation of said afferent neurons innervating said participants;
a sensitization of said afferent neurons innervating said participants;
an activation of the PSN by said participants;
a sensitization of the PSN by said participants;
an activation of said injured afferent neurons including their cell bodies, axons, nerve endings and receptors, and dendrites by PSN;
a sensitization of said injured afferent neurons including their cell bodies, axons, nerve endings and receptors, and dendrites by PSN;
an activation of said disordered afferent neurons including their cell bodies, axons, nerve endings and receptors, and dendrites by PSN;
a sensitization of said injured afferent neurons including their cell bodies, axons, nerve endings and receptors, and dendrites by PSN;
an activation of a second group of afferent neurons by said injured participant;
an activation of a second group of afferent neurons by said disordered participant;
a sensitization of a second group of afferent neurons by said injured participants;
a sensitization of a second group of afferent neurons by said disordered participants;
an activation of a second group of afferent neurons including the activation of their cell bodies, axons, nerve endings and receptors, and dendrites associate with and by said disordered afferent neurons in the peripheral nervous system;
a sensitization of a second group of afferent neurons including the sensitization of their cell bodies, axons, nerve endings and receptors, and dendrites associate with and by said disordered afferent neurons in the peripheral nervous system;
an activation of a second group of afferent neurons including the activation of their cell bodies, axons, nerve endings and receptors, and dendrites associate with and by said activated afferent neurons in the peripheral nervous system;
a sensitization of a second group of afferent neurons including the sensitization of their cell bodies, axons, nerve endings and receptors, and dendrites associate with and by said sensitized afferent neurons in the peripheral nervous system;
a sensitization of a second group of afferent neurons associate with and by said activated afferent neurons in the central nervous system leading to the sensitization of the peripheral endings and receptors of said second group of afferent neurons in the peripheral nervous system;
an activation of a second group of afferent neurons relate to and by said sensitized afferent neurons in the central nervous system leading to the activation of the peripheral endings and receptors of said second group of afferent neurons in the peripheral nervous system;
an activation of a second group of afferent neurons associate with and by said disordered afferent neurons in the central nervous system leading to the activation of the peripheral endings and receptors of said second group of afferent neurons in the peripheral nervous system;
a sensitization of a second group of afferent neurons associate with and by said injured afferent neurons in the central nervous system leading to the sensitization of the peripheral endings and receptors of said second group of afferent neurons in the peripheral nervous system;
a sensitization of a second group of afferent neurons relate to and by said disordered afferent neurons in the central nervous system leading to the sensitization of the peripheral endings and receptors of said second group of afferent neurons in the peripheral nervous system;
an activation of PSN relate to said injured participant;
a sensitization of PSN relate to said disordered participant;
an activation of PSN relate to said injured participants;
a sensitization of PSN relate said disordered participants;
an activation of a second group of afferent neurons including the activation of their cell bodies, axons, nerve endings and receptors, and dendrites by PSN;
a sensitization of a second group of afferent neurons including the sensitization of their cell bodies, axons, nerve endings and receptors, and dendrites by PSN;
an activation of PESN by said participants;
a sensitization of the PESN by said participants;
an activation of said afferent neurons including the activation of their cell bodies, axons, nerve endings and receptors, and dendrites by injured PSN;
a sensitization of the afferent neurons including the activation of their cell bodies, axons, nerve endings and receptors, and dendrites by disordered PSN;
a means for sensitizing the immune system leading to said auto-immune diseases and disorders; and
a means for activating the immune system leading to said auto-immune diseases and disorders.

17. An elucidation of a neurophysiologic basis of rheumatism, degenerative and autoimmune diseases and disorders, specifically, fibromyalgia, fibrositis, myofascial pain syndrome, trigger point, cervicobrachial pain syndrome, palindromic rheumatism, arthropathies, irritable gastrointestinal tract and idiopathic auto-immune soft tissue diseases in a human in whom an injury to, disorder or derangement of the participating tissue or tissues leads to a cascade of events wherein said participating tissues are the tissues of the spine, organs peripheral to said spinal tissues, small, medium and large, unmyelinated and myelinated afferent neurons, preganglionic sympathetic neurons (PESN), postganglionic sympathetic neurons (PSN), interneurons, spinal neurons and immune system and wherein said participating tissues are the participants in the production of said diseases and disorders comprises:
an injury to said participant; a disorder of said participant;
an injury to said participants; a disorder of said participants;
an activation of said afferent neurons associate with said participant;
a sensitization of said afferent neurons associate with said participant;
an activation of said afferent neurons innervating said participant;
a sensitization of said afferent neurons innervating said participant;
an activation of said afferent neurons associate with said participants;
an activation of said afferent neurons innervating said participants;
a sensitization of said afferent neurons innervating said participants;
an activation of the PSN by said participants;
a sensitization of the PSN by said participants;
an activation of said injured afferent neurons including their cell bodies, axons, nerve endings and receptors, and dendrites by PSN;
a sensitization of said injured afferent neurons including their cell bodies, axons, nerve endings and receptors, and dendrites by PSN;
an activation of said disordered afferent neurons including their cell bodies, axons, nerve endings and receptors, and dendrites by PSN;
a sensitization of said injured afferent neurons including their cell bodies, axons, nerve endings and receptors, and dendrites by PSN;
an activation of a second group of afferent neurons by said injured participant;
an activation of a second group of afferent neurons by said disordered participant;
a sensitization of a second group of afferent neurons by said injured participants;
a sensitization of a second group of afferent neurons by said disordered participants;
an activation of a second group of afferent neurons including the activation of their cell bodies, axons, nerve endings and receptors, and dendrites associate with and by said disordered afferent neurons in the peripheral nervous system;
a sensitization of a second group of afferent neurons including the sensitization of their cell bodies, axons, nerve endings and receptors, and dendrites associate with and by said disordered afferent neurons in the peripheral nervous system;
an activation of a second group of afferent neurons including the activation of their cell bodies, axons, nerve endings and receptors, and dendrites associate with and by said activated afferent neurons in the peripheral nervous system;
a sensitization of a second group of afferent neurons including the sensitization of their cell bodies, axons, nerve endings and receptors, and dendrites associate with and by said sensitized afferent neurons in the peripheral nervous system;
a sensitization of a second group of afferent neurons associate with and by said activated afferent neurons in the central nervous system leading to the sensitization of the peripheral endings and receptors of said second group of afferent neurons in the peripheral nervous system;
an activation of a second group of afferent neurons relate to and by said sensitized afferent neurons in the central nervous system leading to the activation of the peripheral endings and receptors of said second group of afferent neurons in the peripheral nervous system;
an activation of a second group of afferent neurons associate with and by said disordered afferent neurons in the central nervous system leading to the activation of the peripheral endings and receptors of said second group of afferent neurons in the peripheral nervous system;
a sensitization of a second group of afferent neurons associate with and by said injured afferent neurons in the central nervous system leading to the sensitization of the peripheral endings and receptors of said second group of afferent neurons in the peripheral nervous system;
a sensitization of a second group of afferent neurons relate to and by said disordered afferent neurons in the central nervous system leading to the sensitization of the peripheral endings and receptors of said second group of afferent neurons in the peripheral nervous system;
an activation of PSN relate to said injured participant;
a sensitization of PSN relate to said disordered participant;
an activation of PSN relate to said injured participants;
a sensitization of PSN relate said disordered participants;
an activation of a second group of afferent neurons including the activation of their cell bodies, axons, nerve endings and receptors, and dendrites by PSN;
a sensitization of a second group of afferent neurons including the sensitization of their cell bodies, axons, nerve endings and receptors, and dendrites by PSN;
an activation of PESN by said participants;
a sensitization of the PESN by said participants;
an activation of said afferent neurons including the activation of their cell bodies, axons, nerve endings and receptors, and dendrites by injured PSN;
a sensitization of the afferent neurons including the activation of their cell bodies, axons, nerve endings and receptors, and dendrites by disordered PSN;
a means for sensitizing the immune system leading to said auto-immune diseases and disorders; and
a means for activating the immune system leading to said auto-immune diseases and disorders.

18. The elucidation according to claim 14 wherein said sensitization of a second group of afferent neurons comprise afferent neurons having A-alpha, A-beta, A-gamma, A-delta and C fibers associate with and by said activated afferent neurons in the central nervous system leading to the sensitization of the peripheral endings and receptors of said second group of afferent neurons in the peripheral nervous system.

19. The elucidation according to claim 14 wherein said sensitization of a second group of afferent neurons comprise afferent neurons having A-alpha, A-beta, A-gamma, A-delta and C fibers including the sensitization of their cell bodies, axons, nerve endings and receptors, and dendrites by PSN.
